(19) United States
(12) Patent Application Publication
(10) Pub. No.: US 2026/0356708 A1
(43) Pub. Date: Nov. 12, 2026

(54) NANOCOMPOSITE FOR SOLUTION DISINFECTION

(71) Applicant: King Abdulaziz University, Jeddah (SA)

(72) Inventors: Khalid A. Alamry, Jeddah (SA); Mahmoud A. Hussein, Jeddah (SA); Hasinah K. Albeladi, Jeddah (SA); Reda M. El-Shishtawy, Jeddah (SA); Raed I. Felimban, Jeddah (SA); Hossam H. Tayeb, Jeddah (SA); Saleh A. Mohamed, Cairo (EG)

(21) Appl. No.: 19/199,789

(22) Filed: May 6, 2025

(51) Int. Cl. C02F 1/50; A01N 25/10; A01N 25/34; A01N 59/00; A01P 1/00; B82Y 5/00

(52) U.S. Cl. CPC C02F 1/50; A01N 25/10; A01N 25/34; A01N 59/00; A01P 1/00; B82Y 5/00; C02F 2303/04; C02F 2305/08

(57) ABSTRACT

A nanocomposite is provided. The nanocomposite includes a poly(aniline-co-anisidine) copolymer matrix; multi-walled carbon nanotubes (MWCNTs); and graphene, wherein the MWCNTs and graphene are dispersed within the copolymer matrix. Methods of preparing the nancomposite using dialdehyde carboxy methyl cellulose (DCMC) are provided. Methods of disinfection a solution using the nancomposite are also provided.

### FIELD OF THE INVENTION

The invention is generally related to nanocomposites useful for disinfecting water by inhibiting growth of bacterial pathogens.

### BACKGROUND OF THE INVENTION

The primary objective of water disinfection is to eliminate germs that cause waterborne illnesses, a method that has been employed for an extended period. Decontamination is the procedure of eliminating dangerous bacteria, cysts, and pathogenic germs by 99.9% by different chemical and physical means. An accessible, hygienic, and sufficient drinking water supply is fundamental to the continued existence of mankind and the welfare of living [1,2]. Notwithstanding advancements in purifying water technology, access to potable healthy freshwater continues to be insufficient in many emerging nations. A significant difficulty in obtaining clean water is bacterial sterilization, since inadequate treatment allows dangerous bacteria to persist in the water availability, therefore facilitating the transmission of illnesses [3-5]. The emergence of multidrug-resistant bacteria in the past few decades exacerbates the challenge of battling these germs. Conventional purifying systems primarily employ membrane filtration to extract bacteria based on size discrepancies, which does not inherently lead to bacterial cell death [6,7]. These devices are susceptible to exterior fouling due to biofilm development and are expensive and challenging to clean, rendering them impractical for reuse. Moreover, the functioning of membrane filtration systems necessitates significant energy expenditure [8-10].

Conducting polymers have garnered significant interest from scientists and engineers due to their unique blend of physical and chemical attributes. Their distinctive features, such as the ability for both chemical and electrochemical synthesis, distinct electronic properties, diverse range, processing advantages akin to conventional polymers, and the potential for cost-effectiveness, make them appealing for various applications. These applications span a wide spectrum, including batteries, capacitors, transistors, photovoltaic cells, light-emitting diodes, aircraft fuselage, and biochemical analysis [11, 12]. Moreover, with the growing environmental considerations, there has been a recent surge in their utilization as film-forming corrosion inhibitors or as coatings with anticorrosion properties to safeguard metallic substrates [13-19]. Polyaniline, recognized as a highly intriguing conducting polymer, has captured significant attention within the scientific community. Its appeal lies in its cost-effectiveness, environmental stability, ease of synthesis through chemical or electrochemical methods, and the ability to tailor its mechanical, electrical, and optical properties for diverse applications [20-25]. A valuable method for enhancing polyaniline's overall properties involves the formation of copolymers with other aniline derivatives. The introduction of side substituents onto polyaniline presents an avenue for improving solubility, process ability, and conductivity while also expanding the overall delocalized system to some extent through the selection of suitable substituents [26]. Poly (o-methoxy aniline) emerges as a compelling derivative of polyaniline, featuring an electron-donating methoxy group as a side substituent to enhance solubility. This modification has proven beneficial in various applications, including chemical sensors and electro chromic displays [27, 28]. Concurrently, extensive efforts have been dedicated to augmenting the surface area of PANI, improving its electrical conductivity, and enhancing the efficiency of electrochemical charging and discharging. This has been achieved through the incorporation of nanostructured materials that exhibit both conductivity and chemical stability, such as carbon nanotubes (CNTs) and graphene [29-32]. Recently, significant attention has been directed towards composites comprising graphene and carbon nanotubes, benefiting from their substantial specific surface area and exceptional electrical conductivity [33-35]. Leveraging their superior electronic characteristics, these composites have found applications in the fabrication of optoelectronic devices, organic solar cells, transparent conducting oxides, electrode materials, and the thermal management of battery packs [36, 37]. While numerous studies have individually explored PANI-CNT and PANI-Gr composites, limited research is available on hybrid PANI-CNT-Gr composites [40-45].

Improved composites that can be used for the disinfection of solutions are needed.

### SUMMARY

An aspect of the disclosure provides a nanocomposite, comprising a poly(aniline-co-anisidine) copolymer matrix; multi-walled carbon nanotubes (MWCNTs); and graphene, wherein the MWCNTs and graphene are dispersed within the copolymer matrix. In some embodiments, the MWCNTs and graphene are uniformly dispersed within the copolymer matrix. In some embodiments, the MWCNTs and graphene comprise 1-10 wt % of the nanocomposite. In some embodiments, the MWCNTs and graphene comprise 1, 3, 5, or 10 wt % of the nanocomposite. In some embodiments, the nanocomposite has a spherical morphology with an average diameter of 0.1-0.5 μm.

Another aspect of the disclosure provides a method of preparing a nanocomposite as described herein, comprising mixing a first solution containing MWCNTs and graphene with a second solution containing aniline (ANI) and o-anisidine (OA) monomers to form a mixture; adding dialdehyde carboxy methyl cellulose (DCMC) to the mixture to form a deposition; filtering and washing the deposition to form the nanocomposite; and drying the nanocomposite.

Another aspect of the disclosure provides a method of disinfecting a solution, comprising contacting the solution with a nanocomposite as described herein under conditions suitable for killing one or more pathogens; and removing the composite from the solution. In some embodiments, the solution is an aqueous solution. In some embodiments, the one or more pathogens are bacterial pathogens. In some embodiments, the one or more bacterial pathogens are selected from S. aureus and E. coli.

### BRIEF DESCRIPTION OF THE DRAWINGS

FIG. 1. Schematic representation of synthesis process of PANI-co-POA-(CNTs-Gr)_{a-d} nanocomposites.

FIG. 2. FT-IR spectra for (i) pure PANI-co-POA, (ii) PANI-co-POA-(CNTs-Gr)ₐ, (iii) PANI-co-POA-(CNTs-Gr)_{b}, (iv) PANI-co-POA-(CNTs-Gr)_{c} and (v) PANI-co-POA-(CNTs-Gr)_{d}.

FIG. 3. XRD patterns for (i) pure PANI-co-POA, (ii) PANI-co-POA-(CNTs-Gr)ₐ, (iii) PANI-co-POA-(CNTs-Gr)_{b}, (iv) PANI-co-POA-(CNTs-Gr)_{c} and (v) PANI-co-POA-(CNTs-Gr)_{d}.

FIGS. 4A-D. SEM micrographs for (a) pure PANI-co-POA, (b) PANI-co-POA-(CNTs-Gr)ₐ and (c-d) PANI-co-POA-(CNTs-Gr)_{c}.

FIGS. 5A-D. TEM images for (a-b) PANI-co-POA-(CNTs-Gr)_{c} and (c-d) PANI-co-POA-(CNTs-Gr)_{d}. nanocomposites.

FIGS. 6A-B. TGA (a) and DTG (b) curves of pure PANI-co-POA and of PANI-co-POA-(CNTs-Gr)_{a-d} at a heating rate 10 °C/min.

FIGS. 7A-F. images of bacterial growth inhibition for control (a, b), PANI-co-POA-(CNTs-Gr)_{c} (c, d), and PANI-co-POA-(CNTs-Gr)_{d} (e, f), nanocomposite materials contacted with S. aureus and E. coli pathogenic bacteria.

### DETAILED DESCRIPTION

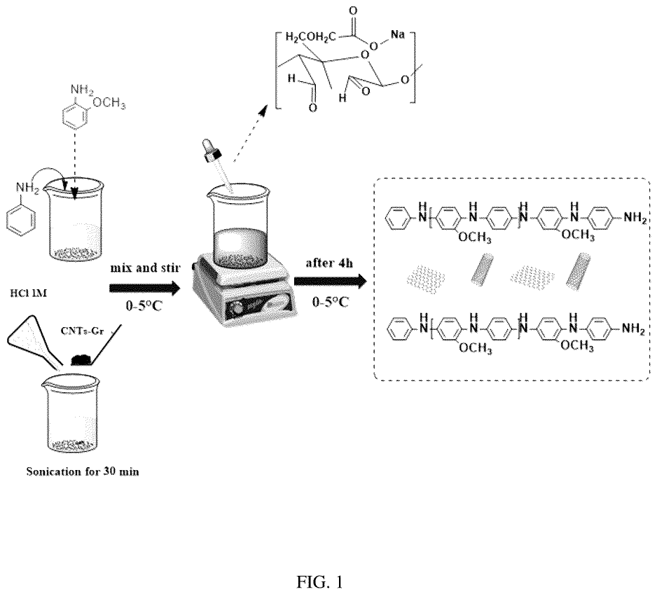

Embodiments of the disclosure provide compositions and methods for inhibiting growth of pathogens in a solution. With reference to FIG. 1, a composite as described herein comprises a poly(aniline-co-anisidine) copolymer matrix; multi-walled carbon nanotubes (MWCNTs); and graphene. The MWCNTs and graphene are dispersed within the copolymer matrix.

Polyaniline (PANI) is a conductive polymer of the semi-flexible rod polymer family having selectivity to certain metal ions due to its multi-redox reactions, excellent electronic properties, and high thermal stability. Polymerized from aniline, polyaniline can be found in one of three oxidation states: leucoemeraldine (fully reduced), emeraldine, and pernigraniline (fully oxidized with imine links instead of amine links). Embodiments include any one of the three states or mixtures of two or three of the states. Polyaniline may be produced, for example, in the form of long-chain polymer aggregates, surfactant (or dopant) stabilized nanoparticle dispersions, or as stabilizer-free nanofiber dispersions. Poly o-anisidine (POA) is a derivative of PANI. O-anisidine is a substituted aniline that is aniline in which the hydrogen ortho to the amino group has been replaced by a methoxy group. The composites described herein comprise a copolymer of PANI and POA, termed poly(aniline-co-anisidine).

Multi-walled carbon nanotubes (MWCNTs) are composed of two or more concentric cylindrical graphene sheet shells which are coaxially arranged around the center with interlayer separation in the middle. The array of cylinders may be separated by approximately 0.35 nm. MWCNTs can have diameters from 2-100 nm and lengths of several micrometers.

The term “graphene” as used herein refers generally to a form of graphitic carbon, in which carbon atoms are covalently bonded to one another to form a two-dimensional sheet of bonded carbon atoms. The carbon atoms may be bonded to one another via sp2 bonds, and may form a 6-membered ring as a repeating unit, and may further include a 5-membered ring and/or a 7-membered ring. In its crystalline form, two or more sheets of graphene may be stacked together to form multiple stacked layers. Generally, the side ends of graphene are saturated with hydrogen atoms.

A nanocomposite described herein may comprise a poly(aniline-co-anisidine) copolymer matrix; multi-walled carbon nanotubes (MWCNTs); and graphene. In some embodiments, the MWCNTs and graphene are uniformly dispersed within the copolymer matrix. The term “uniformly dispersed” refers to a dispersion of a material, substance, compound, etc., in the bulk (continuous) phase (e.g., polymer matrix) such that the bulk phase is substantially uniform in terms of composition, texture, characteristics, properties, etc.

In some embodiments, a composite as described herein may include MWCNTs and graphene from about 1-30 wt %, e.g. about 1-10 wt % of the nanocomposite. In some embodiments, the MWCNTs and graphene comprise 1, 3, 5, or 10 wt % of the nanocomposite. In some embodiments, the nanocomposite has a spherical morphology with an average diameter of 0.1-2 μm, e.g. 0.1-0.5 μm.

Embodiments include methods of preparing a composite as described herein. The composite may be synthesized in the presence of dialdehyde carboxy methyl cellulose (DCMC). The facilitation of aniline (ANI) and o-anisidine (OA) oxidation in the presence of CNTs-Gr by minute quantities of DCMC represents an innovative approach to nanocomposite preparation. The method may comprise mixing a first solution containing MWCNTs and graphene with a second solution containing ANI and OA monomers to form a mixture; adding DCMC to the mixture to form a deposition; filtering and washing the deposition to form the nanocomposite; and drying the nanocomposite.

Embodiments also provide methods for inhibiting growth of pathogens/microorganisms in a solution (i.e. disinfecting a solution), e.g. an aqueous solution, using a composite as described herein. Exemplary sources of water can be any of a ground water source, an industrial source, a municipal source, water source and/or a combination thereof. In some embodiments, the composite is contacted with the solution for at least 15 to 420 minutes, e.g. about 30-120 minutes, before recovering the composite from the solution. In some embodiments, the composite inhibits growth/kills 60% or more, e.g. about 65%, 70%, 75%, 80%, 85%, 90%, 95%, 96%, 97%, 98%, 99% or more pathogens in a solution. In some embodiments, the composite provides for a synergistic growth inhibition/killing of pathogens.

The terms “microorganism” and “microbe” are used interchangeably herein, and refer to an organism that is unicellular or lives in a colony of cellular organisms such as bacteria, fungi, protest, or archea.

In various embodiments, the pathogens/microorganisms are selected from the group consisting of gram-positive bacteria, gram-negative bacteria, fungus, and combinations thereof. The term “gram-positive bacteria” refers to bacterial cells which stain violet (positive) in the Gram stain assay. The Gram stain binds peptidoglycan which is abundant in the cell wall of gram-positive bacteria. In contrast thereto, the cell wall of “gram-negative bacteria” is low in peptidoglycan, thus gram-negative bacteria adopt the counterstain in the gram stain assay. Exemplary bacteria include bacteria of the genus Acinetobacter, Actinomyces, Aeromonas, Bordetella, Borrelia, Brucella, Burkholderia, Campylobacter, Chlamydia, Clostridium, Corynebacterium, Enterococcus, Erwinia, Escherichia, Francisella, Haemophilus, Helicobacter, Klebsiella, Legionella, Leptospira, Listeria, Mycobacterium, Mycoplasma, Neisseria, Pseudomonas, Rickettsia, Salmonella, Shigella, Staphylococcus, Streptococccus, Treponema, Veillonella, Vibrio or Yersinia. In specific embodiments, the bacteria is Staphylococcus aureus or Escherichia coli.

In some embodiments, the fungus is a yeast or a non-yeast fungus. The fungus may be a fungus of the species Candida albicans, Candida tropicalis, Candida (Clasvispora) lusitaniae, Candida (Pichia) guillermondii, Lodderomyces elongisporus, Debaryomyces hansenii, Pichia stipitis, Asperigillus fumigatus, Blastomyces dermatitidis, Cladophialophora bantiana, Coccidioides immitis, Cryptococcus neoformans, Fusarium spp., Microsporum spp., Penicillium marneffei or Trichophyton spp.

The nanocomposite disclosed herein may be reusable such that it is able to exert biocidal effect repeatedly, as it is prevented from absorption by the microbes. By subsequent separation of the nanocomposite from the microbes by, for example, centrifugating a sample solution containing the antimicrobial composition and bacteria cells, the nanocomposite separates from the microbes and remains suspended in solution. Since the composite is not absorbed by the microbes, it does not precipitate together with the bacteria cells, and is able to retain its antimicrobial efficacy after repeated use.

In addition to solution disinfection, the antimicrobial composition disclosed herein may be used for all kinds of environments such as surfaces in private and public areas, where it is beneficial to interfere with Gram-negative or Gram-positive bacteria and/or fungus, in order to prevent and/or treat colonization.

The antimicrobial composition may be used to inhibit growth of microorganisms in an environment alone or together with other materials such as conventional surfactants or detergents, biocides, fungicides, antibiotics, pH regulators, perfumes, dyes or colorants.

The antimicrobial composition may be used as an antibacterial agent for topical use in cleaning and treatment solutions such as disinfectants, detergents, household cleaner and washing powder formulations in the form of a spray or a dispensable liquid. For example, the antimicrobial composition may be applied to windows, floors, clothes, kitchen and bathroom surfaces and other surfaces in the area of food preparation and personal hygiene.

The antimicrobial composition may also be used as antibacterial ingredients in personal hygiene articles, toiletries and cosmetics. Examples of such toiletries include oral hygiene products, which refer to any composition which is used in the mouth in order to promote oral hygiene. These compositions may be in the form of aqueous solutions, for example, a mouth wash composition; or gels, for example toothpaste or dentrifice compositions. In this context, a dentrice refers to a paste, liquid or powder used to help maintain acceptable oral hygiene. Exemplary personal hygiene articles include but are not limited to soaps, shampoos, shower gels, ointments, creams, lotions, deodorants and disinfectants and storage solutions for contact lenses. Examples of cosmetics include, but are not limited to, foundation make-up, eye liner, lip stick, and lip gloss.

The antimicrobial composition may also be used in industrial settings such as ship hulls, paper manufacturing, oil recovery and food processing. The compounds may also be applied to water processing plants or water distribution systems, such as water pipes, water injection jets, heat exchangers and cooling towers.

In some embodiments, the composite is incorporated into a filter for solution disinfection. The filter can be designed in variety of forms, e.g. comprising a candle, a porous block (radial and/or vertical), a filter bed, a packet, a bag and the like.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

Diseases and pathogenetic contamination in water resources pose a significant threat to water quality worldwide. Microorganisms pollute water globally, making it a significant issue. In this study, we introduce hybrid nanofillers composed of Multiwall Carbon Nanotubes

(CNTs) and Graphene (Gr) into conducting polymer nanocomposites derived from aniline (ANI) and o-anisidine (OA) monomers. The synthesis of PANI-co-POA (CNTs-Gr)_{a-d} nanocomposites was achieved through in situ oxidative polymerization, using dialdehyde carboxy methyl cellulose (DCMC) as a green catalyst or green oxidizing agent in a hydrochloric acid medium. The fabricated materials were utilized as an ultra-effective novel water disinfectant material. Various nanocomposites were prepared with fixed mixed ratios of CNTs-Gr at loadings of 1%, 3%, 5%, and 10%. The materials were comprehensively characterized to assess their chemical structure, crystallinity, morphology, and thermal stability using Fourier transform infrared (FT-IR), x-ray diffraction (XRD), scanning electron microscopy (SEM), transmission electron microscopy (TEM), and thermal gravimetric analysis (TGA-DTG), respectively. The FTIR spectra confirmed the successful synthesis of the nanocomposites by displaying characteristic peaks associated with the desired chemical structure. XRD analysis indicated the crystalline nature of the resulting nanocomposites. TEM images supported the even distribution of CNTs-Gr within the copolymer matrix. Additionally, SEM observations illustrated a spherical morphology for these composites, with an average size falling within the range of. 0.1-0.5 μm. Thermal stability, evaluated through TGA, demonstrated that the incorporation of CNTs-Gr nanofillers significantly enhanced the thermal properties of the nanocomposites. The materials were investigated against contaminated water in the presence of both bacteria (S. aureus and E. coli) by using column technique. Approximately 100% of inhibition of the growth of both bacteria in the composition PANI-co-POA-(CNTs-Gr)_{d}.

### Experimental

### Materials and Reagents

Aniline (99%) and o-toluidine (99%) were supplied from Sigma-Aldrich. Both ANI and OA were subjected to fresh distillation and stored in the absence of light. Multiwall carbon nanotubes (CNTs) and graphene (Gr) were sourced from Chen Yuan, China. Hydrochloric acid (HCl), ethylene glycol, and ethanol were procured from Fisher-Chemical. Sodium periodate (NaIO₄) and carboxymethyl cellulose (CMC) were obtained from Merck. All these reagents were utilized in their original form at analytical status. De-ionized water was consistently employed throughout the entirety of this research.

### Preparation of CNTs-Gr

CNTs-Gr preparation involved the combination of CNTs and Gr in a 1:1 mixing percentage through the following steps: A suspension of Gr (100 mg) in deionized water (30 ml) was created by sonicating Gr in an ultrasonic bath for 2 hours. Simultaneously, CNTs (100 mg) were scattered in 30 ml of deionized water and sonicated for 2 hours. Subsequently, the suspensions of Gr and CNTs were mixed through ultra-sonication for an additional 2 hours. The resultant black powder underwent filtration, rinsing with deionized water, and drying at room temperature [46].

### Preparation of DCMC

For the preparation of DCMC, a similar method to the conventional procedure was employed [47]. A solution was formed by dissolving 1 g of MCM in 20 ml of DDW, followed by stirring to achieve a clear solution. Then, 1 g of NaIO₄ (dissolved in 10 ml of DDW) was gradually added to the solution under mixing. The pH of the mixture was modified to 3.0 using a 1M HCl solution. The resulting mixture was mixed at room temperature in darkness for 24 hours. To halt the reaction, 10 ml of ethylene glycol was added and allowed to react for an additional hour. After this, the product solution flowed into a surplus amount of ethanol to make the product, referred to as DCMC. The obtained precipitate was purified, dried at 40 °C until a stable weight was reached, and then prepared for subsequent use.

TABLE 1
Chemical composition and abbreviations for the pure PANI-co-
POA and PANI-co-POA-(CNTs-Gr)_{a-d} nanocomposite materials.

| | Mono- | Mono- | CNTs-Gr |
|---|---|---|---|
| Sample | mer1(ANI) | mer2(OA) | % / (gm) |
| PANI-co-POA | 1 | 1 | — |
| PANI-co-POA-(CNTs-Gr)ₐ | 0.99 | 0.99 | 1%/(0.02) |
| PANI-co-POA-(CNTs-Gr)_{b} | 0.97 | 0.97 | 3%/(06) |
| PANI-co-POA-(CNTs-Gr)_{c} | 0.95 | 0.95 | 5%/(0.1) |
| PANI-co-POA-(CNTs-Gr)_{d} | 0.90 | 0.90 | 10%/(0.2) |

Preparation of PANI-co-POA-(CNTs-Gr)_{a-d}

The in-situ composites were created through the polymerization of ANI and OA, utilizing DCMC as the catalyst, in the existence of CNTs-Gr. A solution containing CNTs-Gr in 1 M HCl was sonicated at room temperature to distribute the CNTs-Gr. Subsequently, ANI and OA monomers were introduced to the CNTs-Gr suspension and mixed for 30 minutes. The DCMC in an HCl solution was then gradually mixed drop wise into the well-stirred reaction mix. The resulting mix underwent magnetic stirring for 4 hours at temperatures ranging from 0 °C to 5 °C. A brown deposition formed, which was filtered and washed multiple times with deionized water (DDW). Following this, the product was dried for 2 hours in a vacuum oven at 80 °C. The PANI-co-POA-(CNTs-Gr)_{a-d} nanocomposites were synthesized with CNTs-Gr loadings of 1%, 3%, 5%, and 10% as illustrated in Table 1. For comparison, pure PANI-co-POA was fabricated using the same procedures, excluding the addition of any nanofiller.

### Disinfection Process

In a small column, sterilized distilled water was loaded with each of S. aureus (1.37×10⁶ CFU/ml) and E. coli (2.13×10⁶ CFU/ml) and kept in the column having the samples (5 column for S. aureus and 5 column for E. coli) for 30 and 120 min. The eluted solution from each column was used to count the number of bacterial cells using serial dilution technique by inoculating it on nutrient agar plates and incubating the plates at 35 °C for 24 h.

### Instruments and Measurements

Fourier transform infrared (FT-IR) spectroscopy analysis was carried out utilizing a Spotlight® 400 within the wavelength range of 4000 to 500 cm⁻¹, employing the KBr disc technique. X-ray diffraction (XRD) patterns were captured using a Bruker D8 Advance diffract meter with Cu-Kα radiation (λ=1.5406 Å, 40 kV, 40 mA) at a scanning pace of 10° min⁻¹. The form and texture were examined using a high-resolution transmission electron microscope (HRTEM, JEOL TEM-2100, Japan) with an accelerating voltage of 250 kV and a magnification of 20 X. Scanning electron microscopy (SEM) was employed to image the set products and explore their morphology. SEM images were acquired utilizing a Zeiss LEO Supra® 55VP Field Emission and SEM Zeiss 1530. Thermogravimetric analysis TGA measurements were conducted using a Shimadzu D60 instrument under an inert nitrogen atmosphere with a heating rate of 10 °C/min within the temperature range from room temperature to 800 °C.

### Results and Discussion

### Fabrication and Characterization

FIG. 1 illustrates a simple representation for the synthetic process for the pure PANI-co-POA and its related nanocomposite materials of different CNTs-Gr loading.

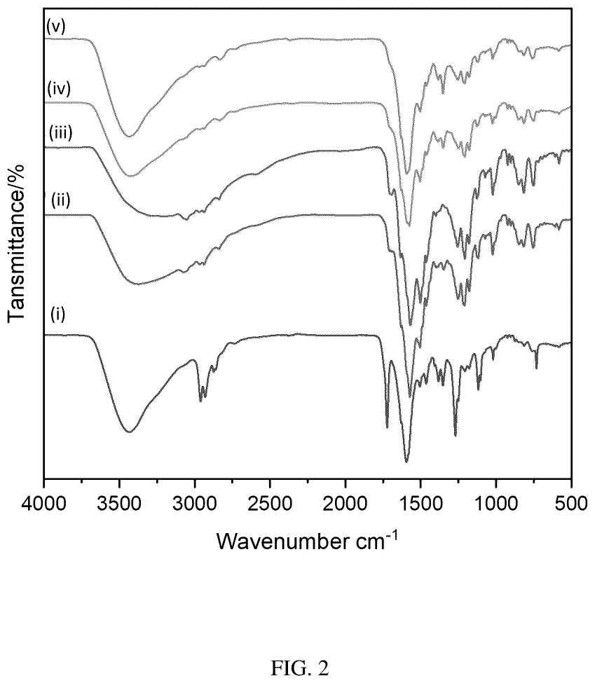

The composition of the products resulting from the oxidative polymerization process was investigated through FT-IR spectroscopy. The FT-IR spectra of both pure PANI-co-POA and PANI-co-POA-(GNTs-Gr)_{a-d} nanocomposites are depicted in FIG. 2. In the spectrum of the copolymer (FIG. 2A), the characteristic bands of PANI-co-POA are evident [48-50]. The peaks at 3442 and 2877 cm⁻¹ in the PANI-co-POA pattern arise from the stretching vibration of N—H from —NH—/—NH₂ and C—H from —OCH₃ in the copolymer. The peaks at 2957 cm⁻¹ are attributed to the C—H stretching vibrations of the aromatic rings. C—N stretching vibrations are observed at 1720 cm⁻¹, and C═C stretching bands for quinonoid rings are noted at 1590 cm-1, while those for benzenoid gatherings appear at 1505 and 1460 cm⁻¹. The peak at 1267 cm-1 is linked to C—N stretching vibration in the units of quinoid imine. The bands at ~1210 and 10¹⁸ cm⁻¹ signify the existence of an o-methoxy group. The band at 1120 cm⁻¹ corresponds to the C—H in-plane and indicates the extent of electron delocalization. The band related to the out-of-plane bending vibration of the C—H bond of p-disubstituted rings is observed at 817 cm⁻¹. The PANI-co-POA-(CNTs-Gr)_{a-d} nanocomposites exhibit small modifications corresponding to the pure PANI-co-POA FT-IR spectrum. Similar peaks are marked in every PANI-co-POA-(CNTs-Gr)_{a-d} nanocomposite, but they all shift to a reduced wavenumber. This shift might be attributed to the interaction between CNTs-Gr nanofillers and the PANI-co-POA chain [41, 51], enhancing the extent of electron delocalization and causing a red shift of distinct peaks. Additionally, the intensity of the C═N peak in the quinoid ring significantly reduces following the incorporation of CNTs-Gr. The spectra of copolymer nanocomposites also show raised peak intensities, becoming either sharper or broader, with a notable increase in the intensity of the peak at 1590 cm⁻¹. The relative intensities of the NH band become broader in the copolymer nanocomposites spectra compared to the FT-IR spectra of the copolymer. Also, the FT-IR spectra of PANI-co-POA-(CNTs-Gr)_{a-d} nanocomposites display characteristic peaks attributed to CNTs-Gr nanofillers [52], confirming the successful formation of the copolymer and copolymer nanocomposites.

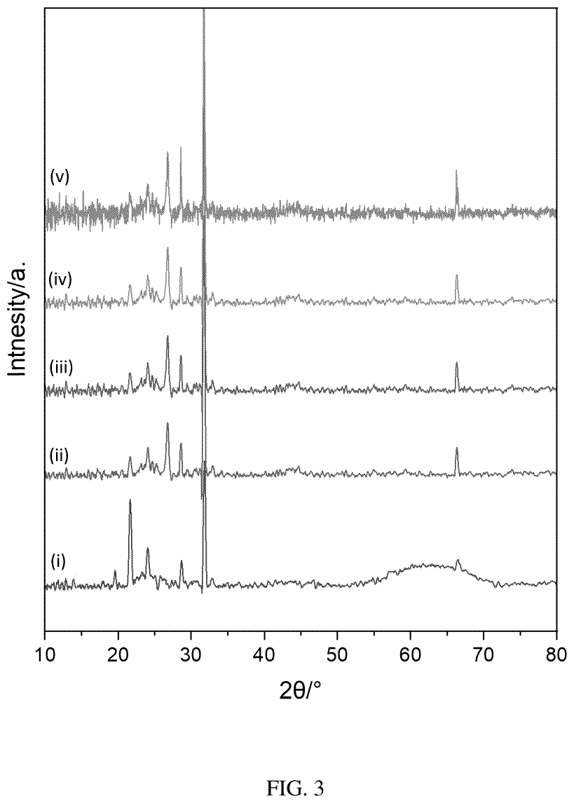

The XRD spectra were employed to analyze the crystalline structures of pure PANI-co-POA and PANI-co-POA-(GNTs-Gr)_{a-d} nanocomposites. FIG. 3 presents the XRD intensities for all specimens in the range of 20 from 10° to 80°. In pure PANI-co-POA, the peak at 21.6° corresponds to the periodicity resemblance to the copolymer chain, while the peak at 23.7° is associated with the periodicity perpendicular to the copolymer chain, representing the distinctive peaks of the copolymer [49, 53, 54]. The XRD spectrum of pure PANI-co-POA exhibits a wide diffraction peak at 58°-66°, offering semicrystalline characteristics of the copolymer. Additional sharp peaks at 19.4°, 28.4°, and 32.1° are also marked in the XRD pattern of pure PANI-co-POA. The XRD patterns of PANI-co-POA-(CNTs-Gr)_{a-d} nanocomposites (curves b-e in FIG. 3) reveal the presence of diffraction peaks related to CNTs-Gr in all samples [52]. The sharp peak at 26.8° corresponds to the (002) crystalline plane of CNTs-Gr, and another peak at 44.3° is associated with graphite. The absence of the third diffraction signal at 23° in the XRD patterns indicates an overlap between the PANI-co-POA peak and the third diffraction signal of CNTs-Gr. In addition, with increasing nanocomposite (CNTs-G) loading, the peak of CNT-G at 26.8° likewise raises, indicating a successful distribution of the filler in the copolymer matrix. The outcomes of XRD verify the impact of CNTs-Gr nanofillers in the nanocomposites. It is noteworthy that the introduction of CNTs-Gr nanofillers led to an increase in the intensity of some copolymer peaks, particularly the diffraction peak at 2θ=32.1°, which became very strong with higher CNTs-Gr content. Additionally, the XRD pattern for the nanocomposites reveals decreased diffraction peaks at 2θ=23.7°. The wide diffraction peak corresponding to PANI-co-POA in the nanocomposites disappears with the introduction of CNTs-Gr nanofillers, suggesting an impact on the crystallinity of the polymer [55]. Furthermore, the diffraction peak of PANI-co-POA at 19.4° is turned to 20.3° in the nanocomposites, showing that the grafted polymer shows various physical and chemical effects corresponding to its parent copolymer.

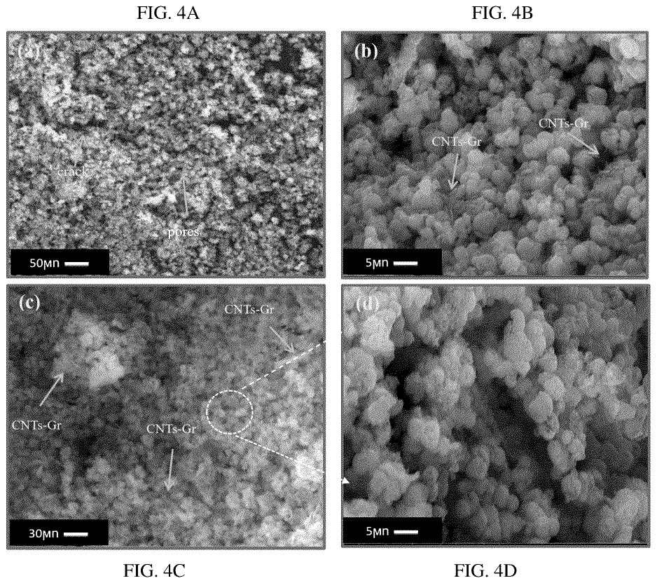

The SEM analysis was conducted to investigate the surface morphology of both the pure copolymer and copolymer-nanocomposites, as illustrated in FIG. 4. FIG. 4a displays surface morphological images of the copolymer without the addition of nanofillers. The SEM images reveal the granular morphology of pure PANI-co-POA, accompanied by randomly distributed cracks. Furthermore, the image of pure PANI-co-POA exhibits high surface roughness with irregular pores. The introduction of CNTs-Gr nanofillers significantly influences the copolymer's morphology. The SEM micrograph of the pure copolymer (FIG. 4a) exhibits a notable diversity in morphology compared to its nanocomposites (FIG. 4b-d). The SEM images of PANI-co-POA-(CNTs-Gr)ₐ and PANI-co-POA-(CNTs-Gr)_{c}, including 1% and 5% wt., respectively, are presented in FIG. 4b-d. These copolymer nanocomposites exhibit a closely packed spherical particulate morphology, representing uniformly scattered spheroids. These globular particles contain numerous polymer chains coiled to form a spherical structure. In addition, the spherical particulate with an average diameter about 0.1-0.5 μm measured with Image, (FIG. 4d). Moreover, the SEM image shows that the PANI-co-POA-(CNTs-Gr)_{a,c} nanocomposites are much smoother than the pure copolymer. The Gr flakes and CNT's tubular structures are uniformly dispersed within the polymer matrix, indicating a strong interaction between the x-bonds of the PANI-co-POA aromatic ring and the graphitic structure of the CNTs-Gr. This interaction should promote the charge-transfer reaction among the two components.

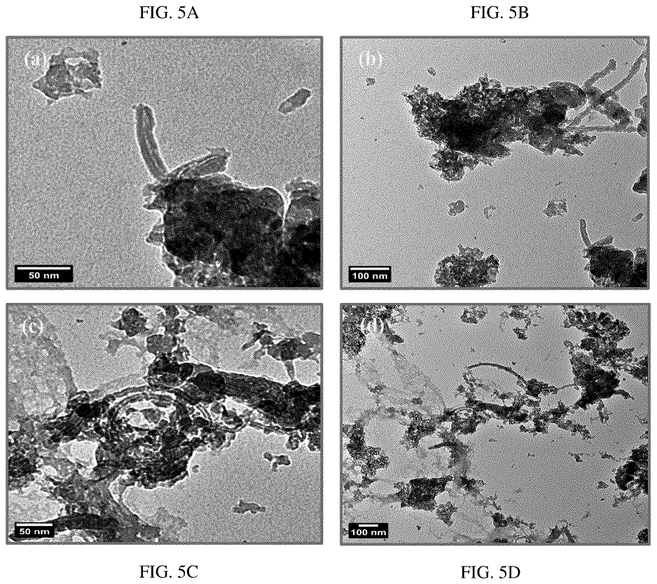

TEM was employed to provide a clearer identification of the morphology of the synthesized materials. The images of TEM of PANI-co-POA-(CNTs-Gr)_{c,d} nanocomposites with 5% and 10% wt loadings are depicted in FIG. 5. The TEM micrographs illustrate that the PANI-co-POA and CNTs-Gr nanofillers have composed a nanocomposite where the nanofillers are embedded in the copolymer matrix. Moreover, CNTs-Gr is evenly distributed on the PANI-co-POA, indicating a robust interaction among the CNTs-Gr and PANI-co-POA. The TEM images of nanocomposites reveal a cluster of solid masses with a uniform surface morphology featuring sharp and pointed edges, suggesting an enhancement in crystallinity for the copolymer nanocomposites.

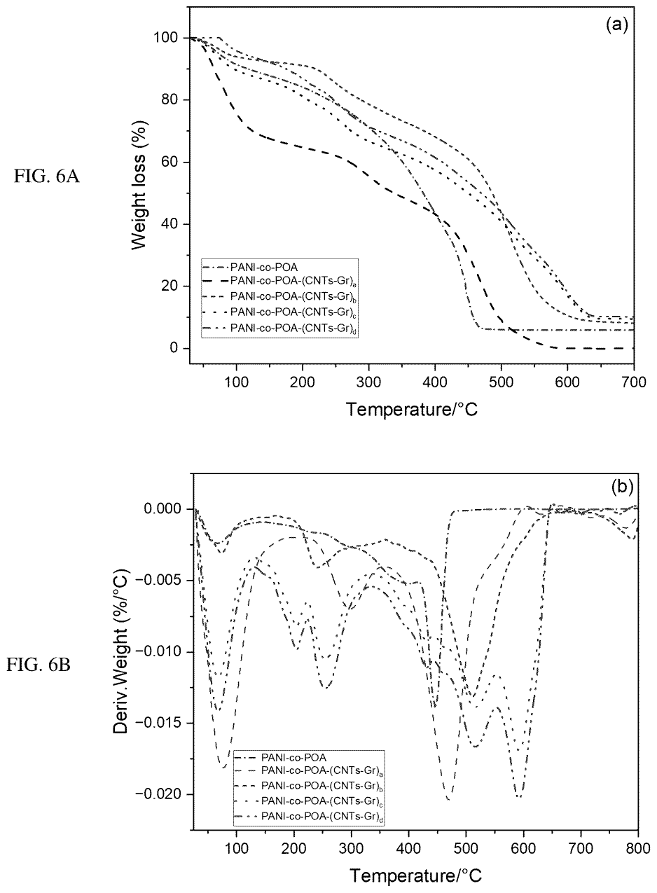

To assess the impact of CNTs-Gr on the thermal characteristics of PANI-co-POA copolymer, TGA and DTG were employed to investigate the stability of the thermal of both pure PANI-co-POA and PANI-co-POA (CNTs-Gr)_{a-d} nanocomposites. FIG. 6 presents a comparison of mass losses through heating in a nitrogen atmosphere at a rate of 10 °C/min for pure PANI-co-POA and PANI-co-POA (CNTs-Gr)_{a-d} nanocomposites.

The TGA curve of the copolymer exhibits a three-step weight loss [56]. The first step, observed at 119 °C, corresponds to the loss of water. The second step, occurring between 119 and 358 °C, corresponds to the loss of dopant HCL. The third step, observed between 358 and 471 °C, corresponds to the final degradation of the copolymer. The thermal behavior of the nanocomposites closely mirrors that of PANI-co-POA, displaying a three-stage decomposition pattern. Nevertheless, it is evident that the thermal stability of the nanocomposites surpasses that of the pure copolymer, and this enhancement is attributed to the presence of CNTs-Gr. The CNTs-Gr nanofillers act as barriers, restraining the degradation of the PANI-co-POA matrix [57-59]. Table 2 summarizes the comparison of five different temperatures for each sample: PDTₘₐₓ (maximum polymer degradation temperature), the final decomposition temperature FDT, and T₁₀, T₂₅, and T₅₀, representing the temperatures at 10%, 25%, and 50% mass loss, respectively. T₁₀ and T₂₅ tend to decrease in nanocomposites compared to pure PANI-co-POA, except for PANI-co-POA (CNTs-Gr), and PANI-co-POA (CNTs-Gr)_{d} nanocomposites.

TABLE 2
Thermal behavior of pure PANI-co-POA and PANI-
co-POA-(CNTs-Gr)_{a-d} nanocomposite materials.

| | | | Temperature (°C) for | | |
|---|---|---|---|---|---|
| | | | various percentage | | |
| | PDTₘₐₓ^{a} | FDT ^{b} | decompositions | | |
| Products | (°C) | (°C) | T10 | T25 | T50 |
| PANI-co-POA | 445 | 471 | 117 | 274 | 379 |
| PANI-co-POA-(CNTs-Gr)ₐ | 468 | 576 | 66 | 100 | 338 |
| PANI-co-POA-(CNTs-Gr)_{b} | 509 | 623 | 219 | 327 | 484 |
| PANI-co-POA-(CNTs-Gr)_{c} | 513 | 634 | 97 | 243 | 441 |
| PANI-co-POA-(CNTs-Gr)_{d} | 516 | 636 | 174 | 272 | 464 |

^{a}Determined from DTG curves.
^{b} The values were determined by TGA at heating rate of 10 °C min⁻¹.

Conversely, T₅₀ exhibits a notable rise with an elevated concentration of CNTs-Gr nanofillers, underscoring the valuable impact of CNTs-Gr on the thermal stability of PANI-co-POA. The order of higher thermal stability at T₅₀ is as follows: PANI-co-POA (CNTs-Gr)_{d}>PANI-co-POA (CNTs-Gr)_{c}>PANI-co-POA (CNTs-Gr)_{b}>PANI-co-POA (CNTs-Gr)ₐ>PANI-co-POA. Furthermore, both FDT and PDTₘₐₓ of PANI-co-POA-(CNTs-Gr)_{a-d} nanocomposites exceed those of pure PANI-co-POA, showing the heightened thermal stability of the nanocomposites.

### Bacterial Disinfection Investigation

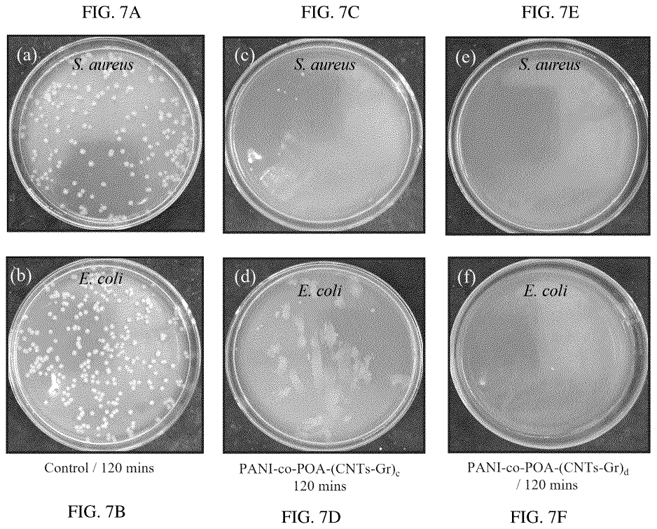

We investigated the percentage of bacterial growth reduction in the fabricated PANI-co-POA and PANI-co-POA-(CNTs-Gr)_{a-d} nanocomposite materials against S. aureus and E. coli using the column tool after 30 and 120 minutes. Table 3 displays the results; while, FIG. 7 displays selected images of bacterial growth inhibition for the control (a, b), PANI-co-POA-(CNTs-Gr)_{c} (c, d), and PANI-co-POA-(CNTs-Gr)_{d} (e, f) nanocomposite materials after 30 and 120 minutes of contact time.

TABLE 3
The bacterial growth inhibition for pure PANI-co-POA and PANI-
co-POA-(CNTs-Gr)_{a-d} nanocomposite materials contacted with S. aureus
and E. coli in column for 30 and 120 min.

| | 30 min | 120 min |
|---|---|---|
| Products | Reduction (%) | Reduction (%) |
| S. aureus | — | — |
| PANI-co-POA | 91.24 | 99.98 |
| PANI-co-POA-(CNTs-Gr)ₐ | 93.71 | 99.56 |
| PANI-co-POA-(CNTs-Gr)_{b} | 94.57 | 99.96 |
| PANI-co-POA-(CNTs-Gr)_{c} | 95.97 | 99.89 |
| PANI-co-POA-(CNTs-Gr)_{d} | 98.39 | 100 |
| E. coli | — | — |
| PANI-co-POA | 97.28 | 99.53 |
| PANI-co-POA-(CNTs-Gr)ₐ | 96.65 | 96.67 |
| PANI-co-POA-(CNTs-Gr)_{b} | 96.54 | 98.83 |
| PANI-co-POA-(CNTs-Gr)_{c} | 99.67 | 99.99 |
| PANI-co-POA-(CNTs-Gr)_{d} | 96.80 | 99.76 |

Reduction % = A (control) − B (treated sample) / A (control)

The results presented in Table 3 demonstrate the effectiveness of the fabricated materials, which varies depending on the filler loading content. The pure PANI-co-POA displays 91.24% after 30 minutes against S. aureus. We achieved a promotional increment by increasing the CNTs-Gr % (93.71-98.39%) for PANI-co-POA-(CNTs-Gr)_{a-d}, respectively. After a long contact period of 120 minutes, all the fabricated samples successfully achieved a bacterial reduction of less than 99.5%. PANI-co-POA-(CNTs-Gr)ₐ was the most efficient among all samples, achieving a 100% reduction. On the other hand, the fabricated materials that were in contact with E. coli after 30 and 120 minutes revealed comparable data with respect to the contact time, which indicates the nature of the bacterial type.

Table 3 shows that when it comes to eradicating bacteria, the materials work far better against S. aureus than E. coli. Using other nanocomposites, prior research has shown that S. aureus could be entirely eliminated, although E. coli remained [60, 61]. The Gram-negative Escherichia coli bacteria has a low negative charge, in contrast to the Gram-positive Staphylococcus aureus, which has a large negative charge [62]. Bacterial adsorption occurs as a result of ion-ion interactions when using positively charged adsorbents such as the PANI-co-POA copolymer and/or its related nanocomposites, PANI-co-POA-(CNTs-Gr)_{a-d}. Moreover, and due to their high aspect ratios, hydrophobic interactions with bacterial cell membranes to remove phospholipids, and production of reactive oxygen near the adsorbed cells to cause oxidative stress, mixed CNTs-Gr (1:1) are effective at killing bacteria [63-65]. Table 4 presents a comparison between some documented studies on pathogen treatment in water and the outcomes achieved. The exceptional efficacy of the synthesized materials in water disinfection was markedly demonstrated utilizing S. aureus in comparison to E. coli. [66-69].

TABLE 4
A comparison for the removal percent of
some previously reported literature with

| | | Removal | Refer- |
|---|---|---|---|
| Adsorbent | Bacteria | (%) | ences |
| Sand/activated carbon | E. coli | 100 | [66] |
| PANI/GN/CNT | S. aureus and E. coli | 99.5/99.2 | [67] |
| Fe₃O₄/SiO₂/NH₂ | E. coli and S. aureus | 97.39 | [68] |
| Ag/Fe₂O₃/Glucose | E. coli | 99 | [69] |
| PANI-co-POA- | S. aureus and E. coli | 100/99.67 | This |
| (CNTs-Gr)_{d} | | | work |

### CONCLUSIONS

A new nanocomposite and synthetic approach for PANI-co-POA-(CNTs-Gr)_{a-d} is presented using a cost-effective, straightforward, environmentally friendly, and concomitant in situ oxidative polymerization approach using green DCMC. This study represents the first instance of introducing DCMC as a dopant for acid-protonating types and the oxidative polymerization of ANI and OA monomers. The materials underwent characterization through various analytical techniques, including FT-IR, XTD, SEM, TEM and TGA-DTG. The FT-IR analysis verified the chemical structure of the copolymer and nanocomposites. The XRD pattern indicated the crystalline nature of the PANI-co-POA-(CNTs-Gr)_{a-d} nanocomposites. TEM analysis demonstrated significant avoidance of common agglomeration problems in CNTs-Gr products, showcasing the nanocomposites in a spherical morphology, as confirmed by SEM. Importantly, the nanocomposites exhibited significantly enhanced thermo gravimetric stability compared to the pure copolymer. The obtained bacterial growth inhibition results indicate the effectiveness of the fabricated materials depending on the filler loading content. Samples were found to exhibit a near-complete suppression of bacterial growth (S. aureus and E. coli) after 120 minutes of interaction with the bacteria in the column.

### Acknowledgment

This project was financially supported by the Deanship of Scientific Research at King Abdulaziz University, Jeddah, under grant no. (IFPP-012-130-2022) and technically by the Innovation and Entrepreneurship Center at King Abdulaziz University.

### REFERENCES

1. Scholz, M., Disinfection. Wetland Systems to Control Urban Runoff. Elsevier, 2006, 155-162.
2. Shannon, M. A., Bohn, P. W., Elimelech, M., Georgiadis, J. G., Marinas, B. J., Mayes, A. M., Science and technology for water purification in the coming decades. In: Nanoscience and Technology: A Collection of Reviews from Nature Journals, 2010, 337-346.
3. Organization, W. H. Drinking-water. Fact sheet. (World Health Organization, 2017). Available from: Drinking-water
4. Schwarzenbach, R. P. et al. The challenge of micropollutants in aquatic systems. Science, 2006, 313:1072-1077.
5. Zhang, M. et al. Magnetically ultraresponsive nanoscavengers for next-generation water purification systems. Nat. Commun., 2013, 4:1866.
6. Willyard, C. The drug-resistant bacteria that pose the greatest health threats. Nature 2017, 543:15.
7. Rotzetter, A. C. C. et al. Combining phosphate and bacteria removal on chemically active filter membrane allows prolonged storage of drinking water. Adv. Mater., 2013, 25:6057-6063.
8. Flemming, H.-C. Biofouling in water systems-cases, causes, and countermeasures. Appl. Microbiol. Biotechnol., 2002, 59:629-640.
9. Pan, J. R., Su, Y.-C., Huang, C. and Lee, H.-C. Effect of sludge characteristics on membrane fouling in membrane bioreactors. J. Membr. Sci., 2010, 349:287-294.
10. Mansouri, J., Harisson, S. and Chen, V. Strategies for controlling biofouling in membrane filtration systems: challenges and opportunities. J. Mater. Chem., 2010, 20:4567-4586.
11. Nalwa, H. S., Handbook of organic conductive molecules and polymers, 1997.
12. Skotheim, T. A., Handbook of conducting polymers. 1997: CRC press.
13. Huerta-Vilca, D., S. R. Moraes, and A. J. Motheo, Electrosynthesized polyaniline for the corrosion protection of aluminum alloy 2024-T₃. Journal of the Brazilian Chemical Society, 2003. 14: p. 52-58.
14. Kalendová, A., et al., Anticorrosion properties of polyaniline-coated pigments in organic coatings. Corrosion Science, 2008. 50(12): p. 3549-3560.
15. Kraljić, M., Z. Mandić, and L. Duić, Inhibition of steel corrosion by polyaniline coatings. Corrosion Science, 2003. 45(1): p. 181-198.
16. Le, D., et al., Corrosion characteristics of polyaniline-coated 316L stainless steel in sulphuric acid containing fluoride. Corrosion science, 2009. 51(2): p. 330-338.
17. Ocon, P., et al., Corrosion performance of conducting polymer coatings applied on mild steel. Corrosion science, 2005. 47(3): p. 649-662.
18. Özyilmaz, A., M. Erbil, and B. Yazici, Investigation of corrosion behaviour of stainless steel coated with polyaniline via electrochemical impedance spectroscopy. Progress in organic coatings, 2004. 51(1): p. 47-54.
19. Santos Jr, J., L. H. C. Mattoso, and A.d.J. Motheo, Investigation of corrosion protection of steel by polyaniline films. Electrochimica Acta, 1998. 43(3-4): p. 309-313.
20. Baker, C. O., et al., Polyaniline nanofibers: broadening applications for conducting polymers. Chemical Society Reviews, 2017. 46(5): p. 1510-1525.
21. Qu, K., et al., Electro-catalytic behavior of multiwall carbon nanotube-doped poly(3, 4-ethylenedioxythiophene) toward environmental pollutant bisphenol A. Journal of The Electrochemical Society, 2019. 166(15): p. H810.
22. Qu, K., et al., A redox conjugated polymer-based all-solid-state reference electrode. Polymers, 2018. 10(11): p. 1191.
23. Qu, K., et al., Carbohydrate-functionalized polythiophene biointerface: design, fabrication, characterization and application for protein analysis. Applied Surface Science, 2019. 486: p. 561-570.
24. Qu, K. and X. Zeng, Ionic liquid-doped polyaniline and its redox activities in the zwitterionic biological buffer MOPS. Electrochimica Acta, 2016. 202: p. 73-83.
25. Zeng, X., K. Qu, and A. Rehman, Glycosylated conductive polymer: A multimodal biointerface for studying carbohydrate-protein interactions. Accounts of chemical research, 2016. 49(9): p. 1624-1633.
26. Tian, Y., K. Qu, and X. Zeng, Investigation into the ring-substituted polyanilines and their application for the detection and adsorption of sulfur dioxide. Sensors and Actuators B: Chemical, 2017. 249: p. 423-430.
27. Profeti, D., L. P. Profeti, and P. Olivi, Effects of electrochemical synthesis conditions on poly (o-methoxyaniline) thin films formation. Materials Chemistry and Physics, 2018. 213: p. 96-101.
28. Yu, Y.-H., et al., Covalent bond-grafted soluble poly (o-methoxyaniline)-graphene oxide composite materials fabricated as counter electrodes of dye-sensitised solar cells. Organic Electronics, 2017. 42: p. 209-220.
29. Kumaraguru, S., J. Yesuraj, and S. Mohan, Reduced graphene oxide-wrapped micro-rod like Ni/Co organic-inorganic hybrid nanocomposite as an electrode material for high-performance supercapacitor. Composites Part B: Engineering, 2020. 185: p. 107767.
30. Li, Y., et al., Facile preparation of polyaniline covalently grafted to isocyanate functionalized reduced graphene oxide nanocomposite for high performance flexible supercapacitors. Colloids and Surfaces A: Physicochemical and Engineering Aspects, 2020. 602: p. 125172.
31. Sumboja, A., et al., Nanoarchitectured current collector for high rate capability of polyaniline based supercapacitor electrode. Electrochimica Acta, 2012. 65: p. 190-195.
32. Viswanathan, A. and A. N. Shetty, Effect of dopants on the energy storage performance of reduced graphene oxide/polyaniline nanocomposite. Electrochimica Acta, 2019. 327: p. 135026.
33. Mittal, G., et al., A review on carbon nanotubes and graphene as fillers in reinforced polymer nanocomposites. Journal of industrial and engineering chemistry, 2015. 21: p. 11-25.
34. Shulaker, M. M., et al., Carbon nanotube computer. Nature, 2013. 501(7468): p. 526-530.
35. Zhang, Y., et al., In-situ synthesis of carbon nanotube/graphene composite sponge and its application as compressible supercapacitor electrode. Electrochimica Acta, 2015. 157: p. 134-141.
36. Wei, Y., Y. Zhu, and Y. Jiang, Photocatalytic self-cleaning carbon nitride nanotube intercalated reduced graphene oxide membranes for enhanced water purification.
Chemical Engineering Journal, 2019. 356: p. 915-925.
37. Wen, L., F. Li, and H. M. Cheng, Carbon nanotubes and graphene for flexible electrochemical energy storage: from materials to devices. Advanced Materials, 2016. 28(22): p. 4306-4337.
38. Hussein, M. A., B. M. Abu-Zied, and A. M. Asiri, The role of mixed graphene/carbon nanotubes on the coating performance of G/CNTs/epoxy resin nanocomposites. Int. J. Electrochem. Sci, 2016. 11: p. 7644-7659.
39. Wegrzyn, M., et al., Thermal and electrical conductivity of melt mixed polycarbonate hybrid composites co-filled with multi-walled carbon nanotubes and graphene nanoplatelets. Journal of Applied Polymer Science, 2015. 132(37).
40. B Kondawar, S., et al., Carbon nanotubes reinforced conducting polyaniline and its derivative poly (o-anisidine) composites. Advanced Materials Letters, 2013. 4(1): p. 35-38.
41. Wang, F., et al., Poly (aniline-co-o-anisidine)/Sulfonated Carbon Nanotubes Composites Prepared by Surface Adsorption Method. Journal of Macromolecular Science, Part B: Physics, 2008. 47(4): p. 743-753.
42. Wang, F., et al., Layer-by-layer assembly of aqueous dispersible, highly conductive poly (aniline-co-o-anisidine)/poly (sodium 4-styrenesulfonate)/MWNTs core-shell nanocomposites. Langmuir, 2008. 24(11): p. 5825-5831.
43. Basnayaka, P. A., et al., Supercapacitors based on graphene-polyaniline derivative nanocomposite electrode materials. Electrochimica Acta, 2013. 92: p. 376-382.
44. Moussa, M., et al., Recent progress and performance evaluation for polyaniline/graphene nanocomposites as supercapacitor electrodes. Nanotechnology, 2016. 27(44): p. 442001.
45. Qu, K., et al., Application of poly (aniline-co-o-methoxyaniline) as energy storage material. Synthetic Metals, 2020. 262: p. 116346.
46. Rovina, K., S. Siddiquee, and S. Md Shaarani, An electrochemical sensor for the determination of tartrazine based on CHIT/GO/MWCNTs/AuNPs composite film modified glassy carbon electrode. Drug and chemical toxicology, 2021. 44(5): p. 447-457.
37. Guo, J., et al., Freezing-thawing effects on the properties of dialdehyde carboxymethyl cellulose crosslinked gelatin-MMT composite films. Food Hydrocolloids, 2013. 33(2): p. 273-279.
48. Butoi, B., et al., Morphological and structural analysis of polyaniline and poly(o-anisidine) layers generated in a DC glow discharge plasma by using an oblique angle electrode deposition configuration. Polymers, 2017. 9(12): p. 732.
49. Mobin, M., R. Alam, and J. Aslam, Investigation of the corrosion behavior of poly (aniline-co-o-anisidine)/ZnO nanocomposite coating on low-carbon steel. Journal of Materials Engineering and Performance, 2016. 25: p. 3017-3030.
50. Rawat, N. K., A. K. Sinha, and S. Ahmad, Conducting poly (o-anisidine-co-o-phenyldiammine) nanorod dispersed epoxy composite coatings: synthesis, characterization and corrosion protective performance. RSC advances, 2015. 5(115): p. 94933-94948.
51. Zengin, H., et al., Carbon nanotube doped polyaniline. Advanced materials, 2002. 14(20): p. 1480-1483.
52. AL-Refai, H. H., A. A. Ganash, and M. A. Hussein, Sensitive and selective voltammetric sensor based on polythiophene nanocomposite mixed MWCNT-G for the determination of Tartrazine. Synthetic Metals, 2021. 280: p. 116875.
53. Pouget, J., et al., Structural investigation of metallic PAN-CSA and some of its derivatives. Synthetic Metals, 1995. 69(1-3): p. 119-120.
54. Sivakumar, K., V. Senthil Kumar, and Y. Haldorai, Zinc oxide nanoparticles reinforced conducting poly (aniline-co-p-phenylenediamine) nanocomposite. Composite Interfaces, 2012. 19(6): p. 397-409.
55. He, J., et al., Conducting polymer and titanium carbide-based nanocomposites as efficient counter electrodes for dye-sensitized solar cells. Electrochimica Acta, 2013. 105: p. 275-281.
56. Sambyal, P., et al., Enhanced anticorrosive properties of tailored poly (aniline-anisidine)/chitosan/SiO2 composite for protection of mild steel in aggressive marine conditions. Progress in Organic Coatings, 2018. 119: p. 203-213.
57. Tjong, S., Y. Meng, and A. Hay, Novel preparation and properties of polypropylene-vermiculite nanocomposites. Chemistry of materials, 2002. 14(1): p. 44-51.
58. Tjong, S., Y. Meng, and Y. Xu, Structure and properties of polyamide-6/vermiculite nanocomposites prepared by direct melt compounding. Journal of Polymer Science Part B: Polymer Physics, 2002. 40(24): p. 2860-2870.
59. Zheming, G., Z. Ling, and L. Chunzhong, Emulsion polymerization: A new approach to prepare graphite oxide coated with polyaniline. Journal of Macromolecular Science®, 2009. 48(2): p. 226-237.
60. Gallaratoa, L. A., Mulko, L. E., Dardanelli, M. S., et al. Synergistic effect of polyaniline coverage and surface microstructuren on the inhibition of Pseudomonas aeruginosa biofilm formation. Colloids Surf B. 2017, 150:1-7.
61. Gizdavic-Nikolaidis, M. R., Bennett, J. R., Swift, S., et al. Broad-spectrum antimicrobial activity of functionalized polyanilines. Acta Biomater. 2011, 7:4204-4209.
62. Li, J., Wang, G., Zhu, H., et al. Antibacterial activity of large-area monolayer graphene film manipulated by charge transfer. Sci Rep. 2014, 4:4359.
63. Carpio, I. E. M., Mangadlao, J. D., Nguyen, H. N., et al. Graphene oxide functionalized with ethylenediamine triacetic acid for heavy metal adsorption and anti-microbial applications. Carbon N Y. 2014, 77:289-301.
64. Prasad, K., Lekshmi, G. S., Ostrikov, K., et al. Synergic bactericidal effects of reduced graphene oxide and silver nano-particles against gram-positive and gram-negative bacteria. Sci Rep. 2017, 7:1591.
65. Tu, Y., Lv, M., Xiu, P., et al. Destructive extraction of phospholipids from Escherichia coli membranes by graphene nanosheets. Nat Nano. 2013, 8:594-601.
66. Pongener, C., Bhomick, P., Upasana Bora, S., Goswamee, R. L., Supong A. and Sinha, D. Int. J. Environ. Sci. Technol. 2017, 14:1897-190.
67. Hussein, M. A., El-Shishtawy, R. M., Alamry, K. A., Asiri, A. M., Mohamed, S. A. ENVIRONMENTAL TECHNOLOGY, 2019, 40:2813-2824.
68. Zhan, S., Yang, Y., Shen, Z., Shan, J., Li, Y., Yang, S., and Zhu, D. J. Hazard. Mater. 2014, 274:115-123.
69. Wei, Z., Zhou, Z., Yang, M., Lin, C., Zhao, Huang, D., Chen Z., and Gao, J. J. Mater. Chem. 2011, 21:16344-16348.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A nanocomposite, comprising
a poly(aniline-co-anisidine) copolymer matrix;
multi-walled carbon nanotubes (MWCNTs); and
graphene,
wherein the MWCNTs and graphene are dispersed within the copolymer matrix.

2. The nanocomposite of claim 1, wherein the MWCNTs and graphene are uniformly dispersed within the copolymer matrix.

3. The nanocomposite of claim 1, wherein the MWCNTs and graphene comprise 1-10 wt % of the nanocomposite.

4. The nanocomposite of claim 3, wherein the MWCNTs and graphene comprise 1, 3, 5, or 10 wt % of the nanocomposite.

5. The nanocomposite of claim 1, wherein the nanocomposite has a spherical morphology with an average diameter of 0.1-0.5 μm.

6. A method of preparing the nanocomposite of claim 1, comprising mixing a first solution containing MWCNTs and graphene with a second solution containing aniline (ANI) and o-anisidine (OA) monomers to form a mixture;
adding dialdehyde carboxy methyl cellulose (DCMC) to the mixture to form a deposition;
filtering and washing the deposition to form the nanocomposite; and
drying the nanocomposite.

7. A method of disinfecting a solution, comprising contacting the solution with the nanocomposite of claim 1 under conditions suitable for killing one or more pathogens; and
removing the composite from the solution.

8. The method of claim 7, wherein the solution is an aqueous solution.

9. The method of claim 7, wherein the one or more pathogens are bacterial pathogens.

10. The method of claim 9, wherein the one or more bacterial pathogens are selected from S. aureus and E. coli.
